(19) United States
(12) Patent Application Publication
(10) Pub. No.: US 2014/0261400 A1
(43) Pub. Date: Sep. 18, 2014

(54) INTRA-ORAL NEBULIZER ACTIVATED BY NEGATIVE INSPIRATORY PRESSURE

(71) Applicant: PNEUMOFLEX SYSTEMS, LLC, MELBOURNE, FL (US)

(72) Inventors: W. Robert ADDINGTON, Melbourne Beach, FL (US); Stuart P. Miller, Indialantic, FL (US)

(73) Assignee: PNEUMOFLEX SYSTEMS, LLC, MELBOURNE, FL (US)

(21) Appl. No.: 13/804,290

(22) Filed: Mar. 14, 2013

(51) Int. Cl. A61M 11/06; A61M 15/00; A61M 16/20; A61M 11/02

(52) U.S. Cl. CPC A61M 11/06; A61M 11/02; A61M 15/009; A61M 16/20
USPC 128/200.21

(57) ABSTRACT

A medication reservoir is contained within a nebulizer body. An air line includes a distal end within the nebulizer body and venturi nozzle. A medication suction line extends from the medication reservoir to the venturi nozzle through which medication is drawn upward and mixed with air after passing through the venturi nozzle and nebulized for discharge through the nebulizer outlet. A valve is positioned within the air line proximal to the venturi nozzle and normally configured in a closed position until a negative inspiratory pressure is applied by a user to open the valve and allow air through the air line and venturi nozzle from a source of air and draw medication upward through the medication suction line for nebulization and discharge through the nebulizer outlet.

### FIELD OF THE INVENTION

The present invention relates to the field of nebulizers, and more particularly, this invention relates to nebulizers having a venturi.

### BACKGROUND OF THE INVENTION

Inhalation is a very old method of drug delivery. In the twentieth century it became a mainstay of respiratory care and was known as aerosol therapy. Use of inhaled epinephrine for relief of asthma was reported as early as 1929, in England. Dry powder inhalers have been used to administer penicillin dust to treat respiratory infections. In 1956, the first metered dosed inhaler was approved for clinical use.

The scientific basis for aerosol therapy developed relatively late, following the 1974 Sugar Loaf conference on the scientific basis of respiratory therapy. A more complete history of the development of aerosol therapy and the modern nebulizer is described in the 2004 Phillip Kitridge Memorial Lecture entitled, “The Inhalation of Drugs: Advantages and Problems by Joseph L. Row; printed in the March 2005 issue of Respiratory Care, vol. 50, no. 3.

Table 8 of the Respiratory Care article, referred to above, page 381, lists the characteristics of an ideal aerosol inhaler as follows:

TABLE 8

Dose reliability and reproducibility
High lung-deposition efficiency (target lung deposition
of 100% of nominal dose)
Production of the fine particles ≦5 μm diameter, with
correspondingly low mass median diameter
Simple to use and handle
Short treatment time
Small size and easy to carry
Multiple-dose capability
Resistance to bacterial contamination
Durable
Cost-effective
No drug released to ambient-air
Efficient (small particle size, high lung deposition)
for the specific drug being aerosolized
Liked by patients and health care personnel

Many standard nebulizers do not achieve a number of these characteristics because they waste medication during exhalation. Further, the particle size is often too large to reach the bottom of the lungs where the medication may be most needed. There is also difficulty in estimating the dose of the drug being given to a patient and there is difficulty in reproducing that dose. There is a possibility of contamination when opening an initially sterile kit, pouring medication into a cup, for example, and assembling the pieces for use by a patient. There is also considerable inefficiency in the medication delivery, with much of it being deposited in the throat, rather than in the lungs.

Venturi type intra-oral nebulizers are disclosed in commonly assigned U.S. Pat. Nos. 7,712,466 and 7,726,306 and U.S. patent application Ser. No. 11/611,425 and published as U.S. Patent Publication No. 2007/0137648, the disclosures which are hereby incorporated by reference in their entirety. These nebulizers overcome some of these drawbacks listed above. These nebulizers are horizontally configured and include a venturi, in one example, at a rainfall chamber. Further enhancements of such nebulizers is desired to achieve even better control over drug delivery to a patient and nebulization.

### SUMMARY OF THE INVENTION

In accordance with a non-limiting example, a nebulizer includes a nebulizer body having a nebulizer outlet. A medication reservoir is contained within the nebulizer body. An air line has a distal end within the nebulizer body and configured as a venturi nozzle. A medication suction line extends from the medication reservoir to the venturi nozzle through which medication is drawn upward and mixed with air after passing through the venturi nozzle and nebulized for discharge through the nebulizer outlet. A valve is positioned within the air line proximal to the venturi nozzle and normally configured in a closed position until a negative inspiratory pressure is applied by a user to open the valve and allow air through the air line and venturi nozzle from a source of air and draw medication upward through the medication suction line for nebulization and discharge through the nebulizer outlet.

In an example, an air flow sensor is positioned within the nebulizer and configured to generate signals indicative of air flow generated by a user's voluntary cough event occurring at nebulization. A processor is configured to receive signals from the air flow sensor and evaluate the involuntary cough event.

In another example, the valve includes a diaphragm and a valve seat and a biasing member that biases the member against the valve seat in a normally closed position. The biasing member comprises a spring, which has a biasing tension that is overcome when a predetermined negative inspiratory pressure is applied to the diaphragm to withdraw the diaphragm from the valve seat and open the valve. The biasing tension is overcome and nebulization begins at a negative inspiratory pressure of −3 cmH₂O to −52 cmH₂O.

In another example, a vane assembly is positioned within the air line and connected to the biasing member and configured to open the valve in response to the negative inspiratory force. A manual valve release member is connected to the diaphragm and configured to allow a user to manually open the valve in another example by moving the diaphragm away from the valve seat. The air line, venturi nozzle and nebulizer outlet may be horizontally oriented when in use. In another example, the venturi nozzle is located to be within a patient's oral cavity when the nebulizer is in use. In another example, the venturi nozzle is positioned in a rainfall chamber. The nebulized medication and air exiting the venturi nozzle may impact a diffuser to aid in nebulization. A secondary suction line is within the rainfall chamber in another example that draws nebulized medication that drops down before discharge through the nebulizer outlet.

### BRIEF DESCRIPTION OF THE DRAWINGS

Other objects, features and advantages of the present invention will become apparent from the detailed description of the invention which follows, when considered in light of the accompanying drawings in which:

FIG. 1 is cross-sectional view of a nebulizer that is activated by negative inspiratory pressure to open a valve and allow nebulization in accordance with a non-limiting example.

FIG. 2 is a fragmentary, sectional view of the nebulizer shown in FIG. 1.

FIG. 3 is a perspective view of another embodiment of the nebulizer in accordance with a non-limiting example.

FIG. 4 is a perspective view of the nebulizer of FIG. 3 with the top housing member as the cover removed and showing basic components of the nebulizer in accordance with a non-limiting example.

FIG. 5 is an enlarged side sectional view of the valve in a closed position in accordance with a non-limiting example.

FIG. 6 is a fragmentary, perspective view in partial cut-away of the closed valve taken along line 6-6 of FIG. 5 in accordance with a non-limiting example.

FIG. 7 is another enlarged side sectional view of the valve similar to that shown in FIG. 5, but showing the valve in an opened position in accordance with a non-limiting example.

FIG. 8 is another fragmentary, perspective view in partial cut-away similar to that shown in FIG. 6 taken along line 8-8 of FIG. 7, and showing the valve in an opened position in accordance with a non-limiting example.

FIG. 9 is another enlarged, side sectional view of the valve similar to that shown in FIG. 5 and showing air feed lines formed within the valve seat body in accordance with a non-limiting example.

FIG. 10 is a perspective end view of the valve shown in FIG. 9.

FIG. 11 is an enlarged side sectional view of the valve similar to that shown in FIG. 9, but showing the valve in an opened position.

FIG. 12 is a fragmentary side sectional view of the nebulizer of FIG. 3 and showing basic components and their relative location to each other in accordance with a non-limiting example.

FIG. 13 is a perspective view of the valve connected to the end of the air line that has the venturi nozzle in accordance with a non-limiting example.

FIG. 14 is a fragmentary plan view of a handheld processing device that can be used in conjunction with the nebulizer for measuring and processing data regarding an involuntary reflex cough event.

FIG. 15 is a block diagram showing example components of the processing device shown in FIG. 14 in accordance with a non-limiting example.

### DETAILED DESCRIPTION OF THE PREFERRED EMBODIMENTS

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

In accordance with a non-limiting example, the nebulizer initiates nebulization upon inhalation by opening a valve contained in the air line proximal to the venturi nozzle and allowing nebulization. The nebulizer is configured as an intra-oral nebulizer, and in one example, is operated for nebulization with half liter air flow using the low pressure air source. Nebulization is activated by a patient breathing and inhaling. Micro amounts of medication are released when required during inspiration and will not flow into the gut because of the low air velocity and the configuration of the nebulizer as an intra-oral nebulizer. This is also aided because the venturi nozzle is positioned intra-orally when the nebulizer is in use. The valve may be a T-valve or other type of valve. The nebulizer delivers one-half (½) liter as an example instead of delivering larger, more uncontrolled dosages such as when using higher pressures of 8-10 psi normal in a hospital or 6-8 psi of a home. The nebulizer is a triggered inspiration that runs into the back of the mouth. It is self-contained. Because most dosages of the nebulized medication go into the lungs upon inhalation, if dangerous drugs are being inhaled during nebulization, it is not likely that they will be released into the ambient and surrounding air to harm others. The nebulizer is not running, e.g., nebulizing, until the medication is nebulized with each inspiration. There is no ambient medication exposure and medication is delivered only during inhalation. The nebulizer may have a variable release pressure and have an external trigger.

There are various mechanics of jet nebulizers that should be understood. A jet nebulizer is a device that is used to deliver medication to the respiratory system using a supplied air source. Traditional nebulizers have a vertical column of air passing through a reservoir of medication, which has a separation at the top of the nozzle allowing the air and medication to mix. This mixture accounts for the initial medication droplet formation due to the drastic change in surface area and aerodynamic effects of the mixture region. This initial droplet formation can be estimated from a linear stability analysis and an aerodynamic loading analysis using parameters such as the Reynolds number, Mach number, and Weber number. This initial droplet formation in this region is normally not sufficient for the desired deposition of the medication in the respiratory tract. To further reduce the droplet size, these droplets travel at high speed and collide with a baffle. This impact energy greatly reduces the droplet size to an acceptable level for deposition of medicine.

This traditional approach has several draw backs. One of the primary factors is that additional medication is required to deliver the proper dose to the desired region of the respiratory tract. Droplet formation occurs outside of the mouth in traditional devices and then has to travel through tubes, masks and the mouth. This additional travel period allows more particle to particle interaction. These particle collisions allow for particle combining, creating a larger diameter. Deposition will not occur with these larger diameter droplets, and therefore waste occurs.

Reducing these particle interactions is possible using the nebulizer 50 as shown in FIG. 1. This nebulizer 50 nebulizes medication to deliver the medication into the stream at the back of the mouth. It operates as a horizontal nebulizer just outside of the mouth to allow for smaller droplet sizes for deposition at a lower zone in the respiratory tract and use less medication, resulting in less waste. The intra-oral venturi nozzle makes much of this possible.

The illustrated nebulizer 50 operates by opening a normally closed valve when a negative inspiratory pressure is applied by a user such as when the patient inhales. This negative inspiratory pressure opens the valve to allow air through the air line and out the venturi nozzle from the source of air such as oxygen connected to the air line and draw medication upward through a medication suction line for nebulization with the air after passing through the venturi nozzle and discharge through the nebulizer outlet. The term air lines could mean channels, communication lines, formed paths in plastic or other communication systems for delivering fluids such as liquid or gases. This valve could be formed as a diaphragm and a valve seat. A biasing member biases the diaphragm against the valve seat in a normally closed position for the valve. The biasing member may be a spring. In another example, a vane assembly is positioned within the air line and connected to the biasing member and configured to open the valve in response to the negative inspiratory pressure.

Referring now to FIG. 1, there is disclosed an improved horizontal nebulizer 50 having a nebulizer body 51 with a venturi nozzle 52. The nebulizer body 51 includes a medication reservoir 58 and a nebulizer outlet 60 configured to be received within an oral cavity of the patient. The nebulizer body is generally horizontally configured and includes a mouthpiece portion 62. In one embodiment, a pacifier housing 64 is added as shown by the dashed line, to form a pacifier or lollipop configuration at the nebulizer outlet. An air line 66 extends horizontally through the nebulizer body and includes at its distal end an outlet formed as the venturi nozzle 52. In this example, a low pressure mixing chamber 68 is formed also at the venturi nozzle into which mediation is delivered and mixed with air flowing out of the narrowed portion of the venturi nozzle 52. FIG. 2 shows in greater detail the air line 66 and venturi nozzle 52, which in this embodiment includes the low pressure mixing chamber 68, which is conically shaped as illustrated.

A medication suction line 70 formed as a channel or other fluid delivery mechanism extends from the medication reservoir 58 to the venturi nozzle 52 and in this example low pressure mixing chamber 68 through which medication is drawn upward and mixed with air after passing through the venturi nozzle 52 and nebulized for discharge through the nebulizer outlet 60. A compressed air line 72 may connect to the end of the nebulizer body via an appropriate fitting 74. As illustrated, the valve 90 is positioned within the air line 66 proximal to the venturi nozzle 52 and normally configured in a closed position until a negative inspiratory pressure is applied by a user and opens the valve to allow air to pass through the air line and through the venturi nozzle from the source of air and draw medication upward through the medication suction line 70 for nebulization and discharge through the nebulizer outlet. As example such as shown in FIG. 5, the valve 90 includes a diaphragm 91 and a valve seat 92 as part of a valve seat body member 92a and a biasing member such as the illustrated spring 93 that biases the diaphragm 91 against the valve seat 92 in a normally closed position for the valve by use of a support rod or other member 94 securing the diaphragm and engaging the spring 93. This spring 93 has a biasing tension that is overcome when a predetermined negative inspiratory pressure is applied to the diaphragm 91 to withdraw the diaphragm from the valve seat and open the valve.

In one example, the biasing tension from the spring or other member is overcome and nebulization begins at a negative inspiratory pressure applied by the user from −3 cmH₂O to −52 cmH₂O. In another example as shown by the dashed lines at 95, a vane assembly is positioned within the nebulizer and connected to an actuator 96 that connects to the biasing member and configured to open the valve in response to the negative inspiratory pressure. The vane assembly 95 may transmit signals to the activator 96, indicative of pressure to open the valve. As shown in the example of FIG. 5, a manual valve release member 97 is connected to the diaphragm 91 and configured to allow a user to manually open the valve such as by pushing the manual valve release member 97 that actuates a pivot or other member to move laterally the diaphragm.

As explained below and referring again to FIG. 1, nebulization begins at a negative expiratory pressure from about −3 cm_{H2}O to about −52 cm_{H2}O. These values can be varied to adjust the amount of inspiratory pressure used to open the valve and begin nebulizer operation. Spring tension can be adjusted or other mechanisms used to adjust this number. The venturi nozzle 52 is positioned at a location to be placed within a patient's oral cavity when the nebulizer is in use and received in the mouth of the user. As illustrated, a rainfall chamber 76 is formed within the body 51 into which the venturi nozzle 52 is formed. As further illustrated, a diffuser 78 acts an impactor upon which the nebulized medication and air exiting the venturi nozzle and low pressure mixing chamber impacts to aid in nebulization. A secondary suction line 80 is formed within the rainfall chamber 76 and draws nebulized medication that had dropped down after impacting the diffuser or impactor 78. A better view of the secondary suction line 80 is shown in FIG. 2. In another example, an airflow sensor 82 can be positioned within the air channel section at the nebulizer outlet and configured to generate signals 83 through a conductor or wirelessly indicative of air flow generated by a patient's involuntary cough event occurring at nebulization. A processor 84 may be associated with the nebulizer or a separate unit such as a handheld unit as shown in FIG. 14. This processor receives signals and evaluates the involuntary cough event as explained in greater detail below.

The dashed lines in FIG. 1 show that the nebulizer outlet can be configured as a infant pacifier 64 and be formed as a housing or lollipop. In another example, it is possible for a housing (not shown) to enclose the body and have an end adjacent to the nebulizer outlet configured as an infant pacifier.

The venturi nozzle 52 is positioned for intra-oral use. In some instances, it may be located outside the oral cavity, but intra-oral location is preferred. The medication is released during breath activation as a horizontal nebulizer compared to an updraft style. Various medications could be mixed during the intake cycle.

In the nebulizer shown in FIG. 1, the flow through the venturi nozzle 52 is not activated until the valve 90 opens in response to a negative inspiratory pressure, such as created from inhalation by the patient. In this nebulizer 50, air pressure is continuous, but nebulization is not. When the negative suction as negative inspiratory pressure opens the valve 90 to allow air flow through the venturi nozzle 52, medication is drawn upward through the medication suction line 70. When this occurs, the nebulized solution extends from the low pressure mixing chamber 68 and impacts the diffuser 78, i.e., impactor and some droplets fall to be picked up by the secondary suction line 80 at the rainfall chamber 76. There are no residual drops, condensation or agglomeration of nebulized medication that forms in front of the rain chamber, which could result in poor nebulization and air being drawn in by the patient. It is recirculated as a true nebulized medication.

In one example, the average pressure to open the valve 90 and begin nebulization occurs at −52 cm with a 2 liter a minute flow rate. It is possible to begin flow at −3 cm negative pressure, but that has been found to be too sensitive. In another example, the nebulizer 50 is configured to open the valve and begin flow at −15 cm corresponding to −1 bar. The nebulizer can be designed to open the valve and begin flow from −3 cm to −100 cm inspiratory pressure. The nebulizer may operate a jet nebulizer. The medication fluid will not be drawn upward and into the airstream until the valve opens to bring air flow through the venturi nozzle. As long as the negative inspiratory pressure is applied, there will be flow. If the negative inspiratory pressure stops, the valve 90 closes and there is no flow. One nebulizer 50 configuration is for a 5 liter per minute air flow, but the nebulizer can be configured for 2 liter up to 15 liter air flow. When the valve 90 opens and the medication begins to flow, the medication impacts the diffuser or impactor 78 and some droplets fall downward and are drawn up by the secondary suction line.

FIG. 3 shows another embodiment of the nebulizer 50″ that includes the nebulizer body 51′ with a top body member forming a cover 100′ and bottom body member 102′ that is included to form the medication reservoir 58′. The air line 66′ is configured to connect to a source of air or oxygen and forming the venturi nozzle 52′. In this example as shown in FIG. 4 with the top body member or cover 100′ removed, the air line 66′ is formed as a polymer material that attaches to fittings on a reservoir cover 104′ that fits over the bottom body member 102′ that is formed to have a medication reservoir 58′ contained within the nebulizer body 51′. The reservoir cover may seal with the bottom body member to seal the medication reservoir. The baffle 78′ is illustrated as part of an extruded portion extending upward from the reservoir cover 104′. The top body member 100′ clips onto the lower body member 102′ to form the complete nebulizer 50′. The valve 90′ in this embodiment is positioned proximal to the venturi before the taper begins as illustrated. These components as illustrated may be formed from suitable polymer and plastic materials.

FIG. 5 shows the valve 90 in a closed position and the biasing member 93 as a spring that biases the diaphragm 91 against the valve seat 92 in a normally closed position. The valve 90 may be formed from first and second body members 90a, 90b that are secured together by threads, adhesive, brazing or other securing techniques. The first housing member 90a is secured onto the end of the taper in the air line 66 that forms the venturi, i.e., venturi nozzle, for the nebulizer such as best illustrated in the example of FIG. 13.

FIG. 6 shows a sectional view of the valve 90 in greater detail and the first body member 90a secured onto the air line and the valve seat 92 that is part of a cylindrical valve seat body member 92a and received within the second housing member. The valve 90 includes the shaft member 94 that extends through an orifice of the valve seat body member 92 and the diaphragm 91 secured on one end and a head formed at the other end that receives the spring 93 as a biasing member as illustrated.

FIG. 7 shows the valve 90 an opened configuration in which a negative inspiratory pressure applied by a user opens the valve 90 to allow air through the air line 66 and pas through the venturi nozzle 52 from the source of air and draw medication upward through the medication suction line 70 for nebulization and discharge through the nebulizer outlet 60.

FIG. 8 is another sectional view of the valve 90, but showing the valve in an opened position. FIG. 9 is another embodiment of the valve 90 and showing additional air orifices 120 formed through the valve seat body member 92a and normally closed off when the valve 90 is closed as shown in FIG. 9, but open to allow air to pass through as shown in FIG. 11. FIG. 10 shows the end view and a better depiction of the orifices 120 in the valve seat body member 92.

FIG. 12 is a fragmentary side sectional view of the nebulizer such as shown in FIG. 3 and showing the basic components with the reservoir cover 104′ positioned over the medication reservoir 58′ to seal the medication reservoir formed in the bottom housing member 102′. The reservoir cover 104′ and its air line supports are illustrated. The baffle 78′ formed in the reservoir cover is also illustrated. As shown, the nebulizer 50′ is formed of four basic components as the air line 66′, top body member 100′ operating as the cover, bottom body member 102′, and reservoir cover 104′. The valve may be secured on the rear of the air line as shown in the embodiment of FIG. 13 or formed inside the air line as shown in the embodiment of FIG. 2.

The outer portion of the housing or body of the pacifier section of the nebulizer such as shown in FIG. 1 may include a section that has a flavoring 106 and a position sensor 108 to indicate the infant's mouth position. This flavoring section is advantageous for sensor placement when an infant sucks on the pacifier or lollipop configured nebulizer. The infant or child will naturally suck on those areas of the pacifier that have the flavoring, indicative that the infant has positioned the pacifier nebulizer in its mouth in the proper position to allow nebulization to occur. When the infant or child has received the pacifier nebulizer in its the proper position as indicated by the sensor 108 indicating this position, the lips or other portion of the infant's mouth covers the position sensor to indicate the proper mouth position. The position sensor sends a signal back to a controller, for example, to activate the nebulizer for operation.

The flavoring 106 on the outer portion of the pacifier allows an infant or child to position the pacifier nebulizer in its proper position in its mouth to allow nebulizer operation, such as pressure to be applied to the air line from an external source, since the infant or child will naturally position the pacifier in a position where it can sense the flavor. A sugar-free flavoring can be used.

When this occurs, the infant will activate the position sensor 108 that indicates the pacifier is in the proper position in the mouth to allow air to be discharged from the air source. Of course, inhalation must occur to open the device for full nebulization and it effects.

Also, the use of more than one medicine container using different medicines can allow simultaneous treatment or delivery of different medicines, actually creating a new drug based upon the combination. It is possible to change the combination depending on infant and child needs. Thus, with the configuration of FIG. 1 an infant can inhale creating the negative inspiratory force to open the valve 90 and activate the nebulizer.

It should also be understood that new medicines can be designed by use of the venturi nozzle 52. It is possible to preload a drug and form a new drug as a method. The nebulizer could operate as a trihaler or quadhaler. It can be placed in a solution in one container as a new drug and combined with a delivery system. It is possible to form the nebulizer and preload the drug. Blow, fill and seal technology could be used to form a throw away nebulizer that is used one time perhaps of the type as shown in FIG. 3 with all plastic components. It could be filled and sealed at the manufacturing line. There could be a prefill port of any different shape or form and different types of medication delivery configurations. An example of different configurations for medicine supply as shown in FIGS. 15 and 16 of the incorporated by reference '466 patent.

The use of a second nozzle can be advantageous because when condensation or agglomeration occurs, a drug will drop down through gravity feed and be redrawn to aid in mixing especially with preloaded medicine. Thus, the nebulizer shown in FIG. 3 can be formed as a sterile preloaded medicated nebulizer as a throw away device. Multiple new drugs can be developed through mixing with the nebulization and a venturi action.

It may also desirable to incorporate a flow meter function such as the in-line design and sensor 82 with the nebulizer configuration shown in FIG. 1 or any pediatric nebulizers. In one design a spinning wheel may be used. The nebulizer can be used to measure involuntary cough and measure the expiatory flow for the voluntary cough and what is the response. This could be beneficial with the pediatric nebulizer using the pediatric nebulizer for diagnoses. A spinning wheel for some type of spirometers could be incorporated into the nebulizer and used with the C5 stimulus, in which the involuntary cough occurs on the average of 4.8 times (average of 5 times) or 4.8 seconds on an average. A spinning wheel can calibrate a processor to measure peak flow and time over the inspiration and expiration and form a graph. It is possible to form the nebulizer where a button is pressed to activate the nebulizer, resulting in an involuntary cough. A flow sensor can be integrated with the nebulizer and measure air flow at the time of the involuntary cough or at the time the button is hit. It is possible to plug the processing device 560 such as the handheld device shown in FIG. 14 into the nebulizer as illustrated. The nebulizer device can perform the pulmonary function test (PFT) that is adequate for use with kids, such as using a lollipop nebulizer. It is possible to measure the velocity of the airflow and draw a graph of the inspiration and expiration over time. The system can draw loop interfaces to the processor or other PC and be compared relative to voluntary cough. During the C5 event it is possible to establish the normal versus the abnormal range.

Reference is made to the commonly assigned and incorporated by reference U.S. Patent Publication Nos. 2011/0040157; 2011/0046653; and 2011/0040211, the disclosures which are hereby incorporated by reference in their entirety. It is possible to diagnose GERD and perform other analysis as explained in those incorporated by reference patent applications, including diagnosing stress urinary incontinence and problems with the lower esophageal sphincter.

It should be understood that different types of air flow sensors can be used besides the spinning wheel configuration. It is possible to design the air flow sensor 82 such as shown in FIG. 1 as a mass air flow sensor that converts the amount of air drawn or expelled into and out of the nebulizer into a voltage signal. Different types of mass air flow sensors could be used such as a vane air flow meter, including using any necessary MEMS technology or using a Karmen vortex or a semiconductor based MAF sensor. It is possible to use a hot wire MAF sensor such as a thermistor, platinum hot wire or other electronic control circuit to measure temperature of incoming air, which is maintained at a constant temperature in relation to the thermistor by an electronic control circuit. As heat is lost, electronic control circuitry can compensate by sending more current through the wire. This is only one example. The wire typically will be kept cool enough such that the temperature does not impact a patient. The hot wire can be placed further into the diffuser and/or main body within the air channel. It is also possible to use an Intake Air Temperature (IAT) sensor.

Another possible air flow sensor is a vane air flow meter that includes basic measuring and compensation plates and other potentiometer circuits. In another example, the air flow sensor uses a “cold wire” system where an inductance of a tiny sensor changes with the air mass flow over that sensor as part of an oscillator circuit whose oscillation frequency changes with sensor inductance. In another example, the flow sensor is an electronic membrane placed in the air stream that has a thin film temperature sensor such as printed on an upstream side and another on the downstream side and a heater in the center of the membrane that maintains a constant temperature similar to the hot-wire. Any air flow causes the membrane to cool differently at the upstream side from the downstream side and this difference indicates the mass air flow. MEMS technology can be used such as MEMS sensors. In this type of sensor, a MEMS sensor has a silicon structure and sometimes combined with analog amplification on a microchip. It includes an analog-to-digital converter on a chip in another example and can be fused with analog amplification and the analog-to-digital converters and digital intelligence for linearization and temperature compensation. The MEMS testing in one example is used for an actuator to control the valve.

It should be understood that although the air flow sensor 82 is shown located at the discharge end of the nebulizer at the diffuser on the exit side of the mixing chamber as shown in FIG. 1, other locations and positions for the air flow sensor or number of air flow sensor members are possible as well as the location of the valve 90.

Air flow can be measured in pounds per second (lbs./sec.) and operate for pulmonary function testing calculations and incentive spirometry use. The nebulizer in this example can work as a differential pressure transducer and connect to a pneumotachygraph (or have a self-contained chip with such function) to record the velocity of respired air. It is possible to process associated data as air flow, air pressure, air resistance, and other Pulmonary Function Testing (PFT) results for respired air and data results from voluntary cough (VC) and involuntary reflex cough testing (iRCT). The pulmonary function testing can use spirometry to assess the integrated mechanical function of the lungs, chest wall and respiratory muscles and measure the total volume of air exhaled from a full lung for total lung capacity and empty lungs as residual volume. The Forced Vital Capacity (FVC) can be measured and a forceful exhalation (FEU can be repeated. Spirometry can be used to establish baseline lung function, evaluate dyspnia, detect pulmonary disease and monitor effects of therapies used to treat respiratory disease and evaluate respiratory impairment and evaluate the operative risk and perform surveillance for occupational-related lung disease. Pulmonary function testing can be used to determine how much air volume is moved in and out of the lungs and how fast the air in the lungs is moved in and out.

This testing can determine the stiffness of the lungs and chest wall for compliance. The flow meter function using the air flow sensor and the associated air flow metering valve together with any processing capability can be used for Inspiratory Muscle Training (IMT) to provide consistent and specific pressures for inspiratory muscle strength and endurance training. The adjustable valve or other adjustable mechanism can ensure consistent resistance and be adjustable such as manually or through microprocessor control for specific pressure settings. It is possible to use the same nebulizer for exercise treatments and therapy and spirometer treatments.

The handheld processing device 560 in FIG. 14 or the processor 80 in FIG. 1 captures the data and can be marketed together with the nebulizer and any necessary catheters for reflex cough testing as a kit. The pneumotachygraph function can be placed in a single chip within the nebulizer or as a separate flow meter device explained below relative to FIG. 14 and connected to the nebulizer 50. Data containing air flow measurement results can be wirelessly transmitted to the handheld processing device or other processor.

The nebulizer 50 may operate in a non-limiting example as a differential pressure transducer. If the nebulizer is to measure voluntary cough or the involuntary reflex cough, an air channel can be connected to the medicine and gas canister (for tartaric acid in one example) and measure the voluntary cough and involuntary reflex cough for in-phase duration from the time from onset to peak and expulsive phase and in-phase volume such as the duration of the glottic closure as explained in greater detail below. It is also possible to measure in-phase peak flow and the expulsive phase peak flow using such device.

A patient (or clinician or physician) can perform a medical treatment with the nebulizer. It is also possible to operate after nebulization to determine airi flow velocity and determine if the patient has improved due to the use and administration of the drug such as the tartaric acid. It is possible to measure and graph results through an air flow sensor as part of the flow meter device and transfer data to the handheld device (or other processing device) and measure flow and pressure over time.

FIG. 14 is an illustration of an exemplary handheld processing device 560. More particularly, it should be understood that this handheld processing device 560 can be used by a nurse practitioner or doctor and receive input as wireless signals for flow meter testing as described above. Also, this handheld processing device 560 can incorporate the circuit and functions as disclosed in the various copending and commonly assigned applications identified above. Catheters and other inputs can be connected to this handheld processing device 560 as explained in the above-identified and incorporated by reference patent applications.

FIG. 15 is a block diagram that illustrates a computer system 500 for the handheld processing device 560. Computer system 500 includes a bus 502 or other communication mechanism for communicating information, and a processor 504 coupled with bus 502 for processing information. Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504.

Computer system 500 may be coupled via bus 502 to a display 512, such as a LCD, or TFT matrix, for displaying information to a computer user. An input device 514, for example buttons and/or keyboard, is coupled to bus 502 for communicating information and command selections to processor 504. Another type of user input device is cursor control, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 operates in response to processor 504 executing one or more sequences of instruction. Execution of the sequences of instructions causes processor 504 to perform the process steps described herein. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to processor 504 for execution. Such a medium may take many forms, including but not limited to, non-volatile media, volatile media, and transmission media. Non-volatile media includes, for example, optical or magnetic disks. Volatile media includes dynamic memory, such as main memory 506. Transmission media includes coaxial cables, copper wire and fiber optics, including the wires that comprise bus 502. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on a magnetic disk of a remote computer. The remote computer can load the instructions into its dynamic memory and send the instructions over a telephone line using a modem. A modem local to computer system 500 can receive the data on the telephone line and use an infrared transmitter to convert the data to an infrared signal. An infrared detector can receive the data carried in the infrared signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

The handheld device 560 preferably uses wireless technology that could include infrared (IR), Bluetooth, or RFID technology for communicating with the wireless transceiver in the wireless module of the nebulizer or a separate wireless interface as illustrated. It can be connected directly also. The handheld processing device 560 includes a wireless module 580 that works in conjunction with the pressure transducer interface and controller 518 and the respiratory air flow sensor (flow meter) interface 581 and sends and receives readings through the antenna 582 or other system that could be used. The wireless module 580 could be located at different locations.

There now follows a general description of physiology for the involuntary reflex cough test (iRCT), which activates the Nucleus Ambiguus. The nebulizer with the flow sensing function may be adapted for measuring both voluntary cough and involuntary reflex cough. The iRCT selectively activates the Medial Motor Cell Column (MMCC) of the spinal cord rather than the (Lateral) LMCC to fire muscles embryologically predetermined to be involuntary cough activated muscles in the pelvis. In the past, urologists did not selectively activate MMCC without overtly activating the LMCC. Magnetic stimulation or electrical spinal cord stimulation activate both cell columns and thus it is not possible to sort out pathology with these. Magnetic stimulation or other approaches from CNS activation set off both columns.

The pelvic muscles that typically are activated with MMCC cough activation include the lumbar-sacral L5/S1 paraspinal axial musculature, which facilitates inpatient continence screening. An example is through MMCC iRCT muscle activation, obtaining L5/S1 paraspinal firing but not L5/S1 lateral gastrocnemius activation because the gastroc muscles are limb muscles activated primarily through the LMCC.

The L-S paraspinals are easier to access with a large pad placed above the sacrum on the midline that contains active, reference and ground combined. It is not important to determine lateralization of the activity like needle EMG for radiculopathy, but only if activation occurs reflexively where the onset latency is under the pressure activation of the abdomen such as the Levator Ani. This is a poor muscle for these purposes because people train it to activate and set their pelvis if the person senses any intra-abdominal pressure elevation. Also, it is difficult to get pads to stick to that area with hair, perspiration, fungal infections or bowel/bladder incontinence present, and other factors.

Some examples have been developed and studied, including a normal CNS patient with Lumax bladder and bowel catheters and pads at L5/S1 paraspinals and a separate EMG machine and electrodes at the pelvic floor in a standard 3:00 and 9:00 o'clock set-up to demonstrate simultaneous involuntary activation with iRCT. This sets off the pelvic floor muscles. Thus, normal airway protection data is obtained and normal CNS data to L1 (where spinal cord ends). The set-up includes a complete T12 that cannot void and needs intermittent catheterization with the same set up, thus demonstrating data for normal airway but no L5/S1 EMG activation by MMCC with all the other data necessary to prove an unsafe bladder by the algorithm. A quadriplegic can demonstrate abnormal airway protection and abnormal EMG activation at both paraspinal and pelvic floor muscles with unsafe bladder measurements that follow the algorithm.

It should be understood that iRCT is an involuntary maneuver that activates embryologically predetermined muscles for airway protection and continence that travel primarily through the MMCC in the spinal cord. Different varieties of lesions are captured and determined with summated interval data approach for general screening purposes.

It is known that the laryngeal cough reflex (LCR) is a strong brainstem-mediated reflex that protects the upper airway by preventing aspiration, or the entrance of secretions, food, and/or fluid into the airway below the level of the true vocal cords (rima glottidis), through elicitation of an involuntary cough. The LCR is activated through the stimulation of cough receptors in the vestibule of the larynx. One way this is achieved is through the inhalation of chemostimulants, such as tartaric acid. Studies have shown that if the LCR is intact, the subject will involuntarily cough (normal LCR) upon inhaling a solution containing TA.

In one non-limiting example, the iRCT involves the inhalation of a nebulized 20% normal saline solution of L-TA (Tartaric Acid). Subjects are asked to perform 1 to 3 effective, full inhalations (about 15-20 second exposure by mouth for tidal breathing wearing a nose clip) from a standard jet nebulizer with at least 50 psi from an oxygen wall unit or tank that produces an average droplet diameter of 1 to 2 microns or less. The nebulizer output is 0.58 mL/min. The initiation of an involuntary cough reflex after any one of the inhalations is the end point of the procedure.

Nebulized TA is a chemical tussive that stimulates irritant receptors in the mucosa of the laryngeal aditus. Mild irritation of these receptors results in nerve impulses being conveyed by the internal branch of the superior laryngeal nerve (ibSLN) to bulbar centers of the brainstem. This nerve constitutes the afferent sensory component of the LCR arc. The efferent component of the LCR is mediated through the vagus, phrenic, intercostals and thoracoabdominal nerves.

Inhaled TA is selective in stimulating rapidly adapting (“irritant”) receptors (RARs), in the supraglottic region. In humans, bilateral anesthesia of the ibSLN abolishes TA-induced cough and permits tidal breathing of the nebulized vapor without coughing, supporting the idea that the RARs are responsible for TA-induced cough.

The physiological response from inhalation of TA in a normal subject is abrupt, forceful coughing of short duration. Using a 20% solution of inhaled nebulized TA is a safe, reliable way to assess the sensation in the supraglottic laryngeal region and subsequently the neurologic circuitry of the LCR. In addition, the ability of the iRCT to predict the integrity of the protective LCR in subjects with stroke has been studied.

A 20% solution of TA as an aerosol causes cough by stimulating sensory nerves in and under the laryngeal epithelium. These nerves have been identified histologically, and the reflexes they cause have been identified. The sensory nerves can be stimulated by both non-isosmolar and acid solutions. Tartaric acid may act in both ways, but the balance between them is uncertain.

The nerves are stimulated by the opening of membrane channels in the nerve terminals. More than 20 categories of channels have now been identified, the opening of which will allow calcium flow into the nerve (and also sodium, with exit of potassium), with the result that an action potential is set up, which travels to the brainstem in the central nervous system (CNS), and reflexively induces cough.

Several different types of sensory nerve ending in the larynx have been identified that may mediate cough and other defensive reflexes. They have been extensively studied, mainly in experimental animals by recording the action potentials in their nerve fibers. The probable candidates for cough are the RARs or ‘irritant’ receptors. These are highly sensitive to mechanical stimuli, to hyperosmolar solutions, and to acids.

Once stimulated, the sensory nerves will induce a variety of defensive reflexes, which protect the lungs from invasion of harmful material. These include cough (an inspiration, followed by a forced expiration against a closed glottis, followed by opening of the glottis with an expiratory blast); the laryngeal cough expiratory reflex (LCER, a powerful expiratory effort with the glottis open); and the glottal closure reflex. In some instances a reflex apnea can be produced. The balance of these reflexes may depend on the nature and the strength of the stimulus. In the case of TA, the LCER seems to be dominant, possibly followed by glottal closure, and the pathophysiological advantage of this response in preventing aspiration is obvious.

There now follows an analysis and test results in greater detail that explain the advantageous use of the involuntary reflex cough test (iRCT) for investigating and diagnosing not only SUI, but also physiological abnormalities such as neurologic deficiencies. The nebulizer as described can be used in conjunction with testing. It should be understood that there are differences between normal and neurological patients.

The EMG from the parineal muscles respond almost simultaneously to the onset of the voluntary cough because the patient does not want to leak. With the involuntary reflex cough test, on the other hand, the fast fibers that are set off reach the abdominal muscles quickly, such as in 17 milliseconds as an example. The patient is not able to set their pelvis. In some of the graphs reflecting urodynamic testing as will be described, it is evident that the onset of the EMG activity does not happen at the same time the pressure rises. Some people that have neuropathy, for example, spinal stenosis or nerve injury (even if it is mild), have a situation that prevents the reflexes from closing before the pressure has changed to push on the bladder. It is not possible to obtain this diagnostic tool methodology unless the involuntary cough reflex test is accomplished. When the involuntary reflex cough test is accomplished, it is possible to demonstrate a latency delay and show that the pathophysiology is a neuropathic problem rather than a structural problem. It is possible to separate the pathophysiology using the involuntary reflex cough test and methodology as described.

In one example, a female patient could have a weak spinal cord and her physiology is normal. This patient may not leak during the test, but the patient cannot protect her airway. Thus, using the methodology apparatus and system associated with the involuntary reflex cough test, in accordance with non-limiting examples, it is possible not only to diagnose an unprotected airway, but also to diagnose normal bladder physiology, including the neurophysiology to the patient's sphincter closure process. This is advantageous because it is then possible to determine when someone cannot protect their airway, even though they may have a normal bladder. Conversely, there are patients with a normal airway, but cannot control their bladder. This process and system as described is able to make that diagnosis and thus the involuntary reflex cough test is an advantageous medical diagnostic tool. For example, it is possible to have a patient with a poorly functioning bladder and normal airway and use of the test allows a doctor to find lower urinary tract symptoms and neuropathology. It becomes possible to diagnose a level of lesion in a patient with a full comprehensive neurologic examination using the involuntary reflex cough test, methodology and apparatus as described.

As will be described in detail later, the various components such as the nebulizer, one or more catheters, any pads for the paraspinal muscles when EMG is used, and drug as part of the nebulizer are inserted in a kit for use at the clinic, hospital or setting. Those components can be discarded after use. The handheld device, of course, will be used again. Use of the kit provides a clinician, doctor or other medical professional the readily available diagnostic tool to determine if a patient has a questionable airway and determine bladder physiology at the same time, all with the use of the one kit.

A kit that is marketed for the iRCT diagnostic tool could include the nebulizer and its drug as TA in one example and one or more pads for the electrodes at the paraspinal and use with EMG. The pad may only be necessary for stress incontinence determinations. A catheter is included in another kit example for use in measuring airway and intra-abdominal pressure. In one non-limiting example, a pad can be placed on a catheter to determine urine leakage and aid in determining stress incontinence. Pressure data is sent to the handheld device in some examples. Obtaining any EMG values from the paraspinal in conjunction with the urology analysis is advantageous. It is possible in one example to measure pressure from a bladder catheter and determine at the same time EMG signals using the EMG electrodes at the L5/S1 in conjunction with the measured involuntary reflex cough test and urology catheter sensing. This is advantageous compared to placing electrodes at the perineal muscles on each side of the sphincter.

It has been found that EMG signals obtained from the perineal muscles have EMG activity from the non-involuntary muscles, i.e., the voluntary muscles blacking out and making analysis difficult because of the signal interference. When the electrodes are placed at the back at the L5/S1 junction, on the other hand, there is nothing else but the paraspinal muscles. It is bone below on each side at the L5/S1 junction. The electrical impulses can be obtained that determine the number of cough impulses coming down through the patient. This is accomplished even if a person has much adipose. The electrode pad used at the L5/S1 junction, in one non-limiting example, typically has an active reference and ground. A pad holds this active reference and ground and the leads as the active reference and ground are plugged into the handheld device (or wireless sensing device in another example) and transmit data to the processor. At least one catheter is also plugged into the handheld device (or wireless sensing device) and measures bladder pressures. A rectal catheter can also be used in some examples. The processor receives EMG signals and determines when the cough event is over.

The involuntary coughs are not hidden by interference when measured from the lower back at the paraspinals as described. This allows a clinician to determine coughs from the bladder when the EMG located at the L5/S1. In one aspect, the area under curve and the average pressure is determined for the cough event corresponding to the involuntary reflex cough test. When this involuntary component of the cough ends, in one example, it becomes silent EMG activity for a period of time. The pressures are at baseline for a period of time, which corresponds in one example to an inhalation. The involuntary component is over.

Sometimes with the involuntary reflex cough test, the cough occurs six times without breathing, but when the patient stops to breathe, the event is over. Using the programming applied with the processor in the handheld device, it is possible to calculate the variables inside the wave as to the involuntary cough and determine airway protection capability. Thus, it is possible to determine and measure cough by defining through appropriate data processing the involuntary cough event compared to the whole cough epoch. For example, a patient could cough ten times, but only the first four are part of the involuntary cough event. The coughs after that event are not part of the epoch.

The programming includes algorithm branches resulting in a conclusion of unsafe bladder based on the data analysis. It is possible to calculate from the waveforms information necessary for assessing airway protection ability. It should be understood that taking the EMG from the L5/S1 is also a better situation for the doctor or clinician, and the patient, since it is more acceptable in a hospital, outpatient or inpatient setting. The doctor or clinician does not have to bend down or stoop and look near the crotch area and place pads since the EMG can now be taken from the paraspinals. Also, the placement of pads and electrodes at the paraspinals is advantageous when patients are standing. If pads are placed at the perineal area, sweat and other problems could cause those pads to become loose and good signals may not be obtained. Also, it should be understood that the perineal muscles do not fire involuntarily. The sphincter may fire involuntarily, but that would create more noise as noted before. Electrodes are not placed at the vagina, but are placed at the paraspinal area instead.

This information obtained from iRct and the EMG taken at the paraspinals allows the doctor or clinician to obtain data leading directly to a diagnosis. For example, some patients that have urinary stress incontinence may have a normal airway in this analysis. It has been found by experimentation that the normal airway is about 50 centimeters water average intra-abdominal pressure. It should be understood that the vesicular pressure (bladder pressure) can track intra-abdominal pressure and terms are often similar and used together. “Bladder” or intravesicular pressure is often used to determine and equate with intra-abdominal pressure. The two are sometimes used interchangeably. Stress urinary incontinence and/or bladder physiology can be diagnosed. The system and method as described leads directly to diagnosis. Fifty centimeters average intra-abdominal pressure over time has been found to correspond to an involuntary reflex cough test normal airway. Thus, the standard deviations or other percentages from that value are used in one non-limiting example to determine an abnormal airway. In a conducted study, the actual value is determined to be about 50.6 centimeters water as compared to voluntary cough values of about 48 centimeters of water. In an outpatient setting, it is possible to have the nebulizer (and drug) and only a pad and test SUI. In hospitalized patients or inpatient settings, this combination is used to measure airway and bladder physiology and the test combination includes a catheter.

It should be understood that the involuntary cough reflex test (iRCT) gives a higher pressure average than obtained using a voluntary cough test. The involuntary cough reflex test is thus a valuable medical diagnostic tool. In one example, four variables are significant in this analysis. These variables include: (1) duration of the event; (2) average intra-abdominal pressure of the event; (3) peak intra-abdominal pressure (max) of the event; and (4) area under the curve. Using these four variables, it is possible to process the received data and obtain a specific diagnosis that could not otherwise be obtained without the use of the involuntary reflex cough test. Individual deficits in a specific variable or combination of variables are used to characterize specific diseases and problems and useful as a medical diagnostic tool.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A nebulizer, comprising:
a nebulizer body comprising a nebulizer outlet;
a medication reservoir contained within the nebulizer body;
an air line having a distal end within the nebulizer body and configured as a venturi nozzle;
a medication suction line extending from the medication reservoir to the venturi nozzle through which medication is drawn upward and mixed with air after passing through the venturi nozzle and nebulized for discharge through the nebulizer outlet; and
a valve positioned within the air line proximal to the venturi nozzle and normally configured in a closed position until a negative inspiratory pressure is applied by a user to open the valve and allow air through the air line and venturi nozzle from a source of air and draw medication upward through the medication suction line for nebulization and discharge through the nebulizer outlet.

2. The nebulizer according to claim 1, wherein said valve comprises a diaphragm and a valve seat and a biasing member that biases the diaphragm against the valve seat in a normally closed position.

3. The nebulizer according to claim 2, wherein said biasing member comprises a spring.

4. The nebulizer according to claim 3, wherein said spring has a biasing tension that is overcome when a predetermined negative inspiratory pressure is applied to the diaphragm to withdraw the diaphragm from the valve seat and open the valve.

5. The nebulizer according to claim 4, wherein the biasing tension is overcome and nebulization begins at a negative inspiratory pressure from −3 cmH₂O to −52 cmH₂O.

6. The nebulizer according to claim 2, and further comprising a vane assembly positioned within the air line and connected to said biasing member and configured to open the valve in response to the negative inspiratory force.

7. The nebulizer according to claim 2, and further comprising a manual valve release member connected to said diaphragm and configured to allow a user to manually open the valve by moving the diaphragm away from the valve seat.

8. The nebulizer according to claim 1, wherein the air line, venturi nozzle and nebulizer outlet are horizontally oriented when in use.

9. The nebulizer according to claim 1, wherein the venturi nozzle is located to be within a patient's oral cavity when the nebulizer is in use.

10. The nebulizer according to claim 1, and further comprising a rainfall chamber into which the venturi nozzle is positioned.

11. The nebulizer according to claim 10, and further comprising a diffuser upon which the air exiting the venturi nozzle and mixed with the nebulized medication to aid nebulization.

12. The nebulizer according to claim 11, and further comprising a secondary suction line within the rainfall chamber that draws nebulized medication that drops down before discharge through the nebulizer outlet.

13. A nebulizer, comprising:
a nebulizer body comprising a nebulizer outlet;
a medication reservoir contained within the nebulizer body;
an air line having a distal end within the nebulizer body and configured as a venturi nozzle;
a medication suction line extending from the medication reservoir to the venturi nozzle through which medication is drawn upward and mixed with air after passing through the venturi nozzle and nebulized for discharge through the nebulizer outlet;
a valve positioned within the air line proximal to the venturi nozzle and normally configured in a closed position until a negative inspiratory pressure is applied by a user to open the valve and allow air through the air line and venturi nozzle from a source of air and draw medication upward through the medication suction line for nebulization and discharge through the nebulizer outlet;
an air flow sensor positioned within the nebulizer and configured to generate signals indicative of air flow generated by a user's involuntary cough event occurring at nebulization; and
a processor configured to receive signals from the air flow sensor and evaluate the involuntary cough event.

14. The nebulizer according to claim 13, wherein said valve comprises a diaphragm and a valve seat and a biasing member that biases the diaphragm against the valve seat in a normally closed position.

15. The nebulizer according to claim 14, wherein said biasing member comprises a spring.

16. The nebulizer according to claim 15, wherein said spring has a biasing tension that is overcome when a predetermined negative inspiratory pressure is applied to the diaphragm to withdraw the diaphragm from the valve seat and open the valve.

17. The nebulizer according to claim 16, wherein the biasing tension is overcome and nebulization begins at a negative inspiratory pressure from −3 cmH₂O to −52 cmH₂O.

18. The nebulizer according to claim 14, and further comprising a vane assembly positioned within the air line and connected to said biasing member and configured to open the valve in response to the negative inspiratory force.

19. The nebulizer according to claim 13, and further comprising a manual valve release member connected to said diaphragm and configured to allow a user to manually open the valve by moving the diaphragm away from the valve seat.

20. The nebulizer according to claim 13, wherein the air line, venturi nozzle and nebulizer outlet are horizontally oriented when in use.

21. The nebulizer according to claim 13, wherein the venturi nozzle is located to be within a patient's oral cavity when the nebulizer is in use.

22. The nebulizer according to claim 13, and further comprising a rainfall chamber into which the venturi nozzle and low pressure mixing chamber are positioned.

23. The nebulizer according to claim 22, and further comprising a diffuser upon which the nebulized medication and air exiting the venturi nozzle and low pressure mixing chamber impacts to aid nebulization.

24. The nebulizer according to claim 23, and further comprising a secondary suction line within the rainfall chamber that draws nebulized medication that drops down before discharge through the nebulizer outlet.
